(19) United States
(12) Patent Application Publication
(10) Pub. No.: US 2024/0043204 A1
(43) Pub. Date: Feb. 8, 2024

(54) SIMPLIFIED SYSTEM FOR COLLECTING, STORING AND TRANSPORTING FERMENTABLE WASTE

(71) Applicant: Bio Tank, Pont-Du-Casse (FR)

(72) Inventors: François-Xavier Postel, Pont-Du-Casse (FR); Xavier Hindelang, Mercy-Le-Bas (FR)

(21) Appl. No.: 18/257,044

(22) PCT Filed: Dec. 10, 2021
(86) PCT No.: PCT/FR2021/052281

§ 371 Date: Jun. 12, 2023

(30) Foreign Application Priority Data
Dec. 11, 2020 (FR) FR2013038

(51) Int. Cl. B65F 1/10; B65F 1/14; B09B 3/40; B65F 1/12

(52) U.S. Cl. CPC B65F 1/10; B65F 1/1405; B09B 3/40; B65F 1/12; B09B 2101/70

(57) ABSTRACT

A removable device for collecting, storing and transporting waste, comprises, in a flow direction of the waste: a device for delivering the waste, a hopper, a device for transferring the materials received in the hopper, a storage tank arranged to store the transferred materials and operating anaerobically in a storage configuration, and arranged to receive the transferred materials in a filling configuration, a device for emptying the tank, which device is arranged to empty the storage tank, the hopper being arranged to direct the waste from the delivery device to the transfer device, the transfer device being arranged to directly transfer the materials received from the hopper to the storage tank, the transfer device being at a positive pressure in the flow direction of the waste.

### CROSS-REFERENCE TO RELATED APPLICATIONS

This application is a national phase entry under 35 U.S.C. § 371 of International Patent Application PCT/FR2021/052281, filed Dec. 10, 2021, designating the United States of America and published as International Patent Publication WO 2022/123188 A1 on Jun. 16, 2022, which claims the benefit under Article 8 of the Patent Cooperation Treaty to French Patent Application Serial No. FR2013038, filed Dec. 11, 2020.

### TECHNICAL FIELD

The present disclosure relates to a device for collecting, storing and transporting fermentable waste.

### BACKGROUND

Fermentable waste is residue of animal or plant origin composed of biodegradable organic material. It can be broken down by microorganisms, for which it represents a power source. Several types of fermentable waste exist: food residue, derived from products of animal origin meat, milk, cheese, eggshells; food scraps of plant origin, such as vegetable peels, fruits peels, used vegetable oils, coffee, tea; green biowaste or yard waste such as lawn clippings, dead leaves, manure, straw; and residue composed of papers and cardboard.

Putrescible waste is residue of animal or plant origin that can degrade spontaneously and within a very short time, without the addition of microorganisms. This is biowaste that can be readily exploited. Not all fermentable waste is necessarily putrescible. Conversely, all putrescible waste is fermentable waste. The difference between putrescible waste and fermentable waste lies in the fact that not all fermentable materials necessarily have the capacity to degrade spontaneously. Indeed, cardboard, paper and certain textiles of organic origin require the addition of specific microorganisms in order to decompose.

Fermentable waste is capable of being treated by composting or methanization. Green waste, kitchen waste or vegetable garden waste are examples of fermentable waste.

Composting is an aerobic biological process for converting and reusing organic matter into a stabilized, hygienic product, similar to a fertile soil, rich in humic and mineral compounds, compost.

Methanization (or anaerobic digestion) is a natural biological process of degradation of anaerobic organic matter, that is, in the absence of oxygen. It occurs naturally in certain sediments, swamps, rice paddies, landfills, and also in the digestive tract of certain animals: insects (termites) or vertebrates (ruminants, etc.). Part of the organic matter is degraded in methane, and another part is used by the methanogenic microorganisms for their growth and reproduction. The decomposition is not complete and leaves the digestate. Methanization is a technique implemented in a digester, also called biogas reactor or biogas plant, wherein the process is accelerated and maintained in order to produce a usable methane, called biogas, or even biomethane after purification. Thus, fermentable waste, and, in particular, organic waste (or products from solid or liquid energy crops), can provide energy.

Various forms and sizes of digesters exist. In one of the rudimentary techniques used, the digester is in the form of a hermetic pit into which black water, sludge, and additional organic compounds allowing the digestion to be facilitated are poured. The gas forms in the sludge and rises to the surface, mixing the sludge by this process. The digested sludge accumulated at the bottom forms part of the digestate and can be drained and used as fertilizer.

Prior to the composting or methanization of the fermentable material, it is necessary to store and transport the fermentable material to one or several places where the latter can be treated, either by composting or by methanization.

Document LU92556 discloses a system for storing and transporting fermentable waste with a fermentable waste storage device including a container for storing waste, a pump for establishing and maintaining a reduced pressure in the container, a mechanical storage facility for storing the container and the pump, and a transporter for transporting the container and the pump to and from the storage facility. According to the patent document, the implementation of a pump for establishing and maintaining a reduced pressure in the container has the advantage of reducing the amount of oxygen within the container, and thus of slowing down the decomposition of the waste. The nuisances associated with the decomposition may thus be delayed, and the waste collection frequency can be reduced. Nevertheless, using the pump to establish a reduced pressure in the container causes the inlet and outlet orifices to be suctioned. In order to be able to add the ground fermentable material to the storage tank, which is under a slight vacuum, it is necessary to stop the pump and to introduce oxygen into the tank to regain atmospheric pressure. The introduced oxygen accelerates the decomposition of the ground materials in the storage tank. Furthermore, it is then necessary to use the pump again to place the storage tank under a slight vacuum. Either the pump flow rate is high and the effluent filtering means is then ineffective, since the gas flow rate passing through it is not nominal, but too high, or the pump flow rate is low, and it is then necessary to wait a long time, that is, on the order of a few hours depending on the volume transferred, in order to be able start introducing ground fermentable materials into the storage tank again.

Document LU92859 discloses a device for storing and/or transporting organic waste comprising a hermetic tank intended to receive the waste; a vacuum pump fluidly connected to the waste tank and configured to suck the gases present in the tank and to create a vacuum in the tank; and a waste discharge chute, fluidly connected by a pipe to the waste tank. The device further includes a unit for neutralizing odors emitted by the gases sucked by the vacuum pump. The document notes that in practice, however, slowing down the decomposition of the waste is not particularly useful. Indeed, when the device is transported by a truck and if the truck is used effectively on a collection round, the truck moves from place to place to collect the waste over a reduced period of time, typically one or several days, until it is full. However, this waste does not decompose in a few days, even in high heat. Once the tank has been filled, the truck can then go to empty its contents at an organic waste treatment center. The storage device described in the document also implements the vacuum pump to transfer the waste from a pipe of the device to the inside of the tank. The problems mentioned above related to the use of a vacuum pump are still present.

Document LU100173 discloses a device for storing and/or transporting organic waste, comprising a hermetic tank intended to receive the waste; a waste discharge chute, connected by a pipe to the waste tank and means for discharging the gases present in the tank. The device includes a unit for neutralizing odors emitted by the gases, fluidly connected to the tank by the discharge means and comprising at least one column of aqueous solution, the odor-neutralizing unit being configured to receive the gases from the tank in the bottom of the column(s). The discharge of the waste and its transfer from the supply pipe to the hermetic tank can be carried out by suction or by a worm. The main problem related to implementing at least one washing column lies in developing the assay of the acidic and basic reagents to be placed in the columns. Indeed, the latter strongly depend on the nature of the waste that will be inserted into the tank, which by definition is unknown before its use. To be effective, the diffusion rate in the washing columns must be slow and constant (bubbling). However, this is not the case when a significant influx of gas occurs, for example, when transferring the contents of a garbage can from the grinder to an almost full tank or when a powerful vacuum pump is used that will not allow the bubbling of the effluent in the washing columns.

There is a tremendous deposit of fermentable waste in packaged products. This is, for example, the case of dairy products and certain convenience foods. It is understood that the use of a grinder is delicate, as the tank for receiving the ground products then includes chips mixing ground fermentable waste and inert chips.

### BRIEF SUMMARY

One aim of the present disclosure is notably to remedy all or part the aforementioned drawbacks.

The present disclosure relates to, in particular, the treatment of waste including putrescible fermentable waste. It can also be applied to the treatment of non-putrescible fermentable waste.

According to a first aspect of the present disclosure, there is proposed a removable device for collecting, storing and transporting fermentable waste including, in a flow direction of the waste:
a device for delivering fermentable waste;
a hopper;
a device for transferring the materials received in the hopper;
a sealed storage tank arranged to store the transferred materials and operating anaerobically in a storage configuration, and arranged to receive transferred materials in a filling configuration; and
a device for emptying the tank arranged to empty the storage tank.

With the present disclosure, the device does not include a grinder; the waste is transferred directly to the storage tank. This allows treatment of waste comprising packages. The removable device can receive more waste, requires less maintenance and is less expensive to produce.

The hopper is arranged to direct the fermentable waste from the delivery device to the transfer device, the transfer device being arranged to transfer the received materials to the storage tank.

The device may additionally include an effluent filtering device (fluidly) connected to the storage tank. The effluent filtering device can be arranged (fluidly connected) between the storage tank and the effluent discharge device. The effluent filtering device may be a solid filtration medium, preferably a charcoal filter or a biofilter.

The device may additionally comprise an effluent storage cylinder connected to the storage tank.

According to the first aspect of the present disclosure, the transfer device can be under positive pressure in the flow direction of the fermentable waste. The positive pressure transfer device may be, for example, a worm or a peristaltic pump.

The delivery device may include a manual opening, preferably a slide or drum valve.

The delivery device may include a container lifter, preferably electrically or hydraulically actuated, arranged to empty a rolling bin.

The delivery device may be of the gravity type.

The hopper may be provided with a nonstick coating of polytetrafluoroethylene (PTFE) or polypropylene (PP) type. Polypropylene has a very low coefficient of friction and good resistance to acids and bases.

The transfer device may comprise a gooseneck cylinder, or pipe, which may be connected above the tank.

The storage tank may be made of a polymer, soft metal or stainless steel material.

The storage tank may be covered, at least partially, with a protective and/or reinforcing layer, for example, of the PTFE, stainless steel or epoxy sheet type.

The storage tank may include wave-breaking discs arranged vertically.

The storage tank may include level sensors.

The storage tank may further include heating means arranged to heat the stored materials.

The device according to the first aspect of the present disclosure may further include control means configured to control the heating means.

The device according to the present disclosure may include an odor trap arranged upstream of the hopper, in the flow direction of the fermentable materials. The odor trap may be formed by a hopper cover provided with a gasket. When closed, the cover retains the odors from the residues of waste that has not been conveyed into the storage tank.

The device according to the present disclosure may include a second odor trap arranged between the transfer device and the storage tank. The second odor trap may be formed by a knife gate valve. The second odor trap makes it possible to ensure that the stored materials cannot return to the hopper. This assembly is particularly advantageous when the transfer device has a gooseneck pipe.

The device according to the present disclosure may further include a pressure reducer arranged between the storage tank and the effluent filtering device.

The device according to the present disclosure may further include a device for discharging effluent generated by the stored material and/or fermentable waste fluidly connected to the storage tank.

The emptying device may be by gravity or suction, for example, by means of a back door or an agricultural valve.

According to a second aspect of the present disclosure, there is proposed a transporter equipped with a device according to the first aspect of the present disclosure, or one or several of its improvements, from a storage facility wherein the device is stored to another storage facility.

The transporter may be a truck. The type of transporter used may depend on the size and shape of the device, and the terrain over which the container is to be transported.

### BRIEF DESCRIPTION OF THE DRAWINGS

Other advantages and particularities of the present disclosure will become apparent on reading the detailed description of implementations and embodiments that are in no way exhaustive, with reference to the appended drawings, in which:

FIG. 1 schematically shows a first embodiment of a device for treating fermentable waste according to the present disclosure;

FIG. 2 schematically shows a detail of the device shown in FIG. 1;

FIG. 3 schematically shows another detail of the device shown in FIG. 1; and

FIG. 4 schematically shows still another detail of the device shown in FIG. 1.

### DETAILED DESCRIPTION

Since the embodiments described below are in no way limiting, it will be, in particular, possible to consider variants of the present disclosure comprising only a selection of the features described, subsequently isolated from the other features described, if this selection of characteristics is sufficient to confer a technical advantage or to differentiate the present disclosure from the prior art. This selection includes at least one feature, preferably functional, without structural details, or with only a portion of the structural details if this part only is sufficient to confer a technical advantage or to differentiate the present disclosure from the prior art.

In the figures, an element appearing in several figures retains the same reference.

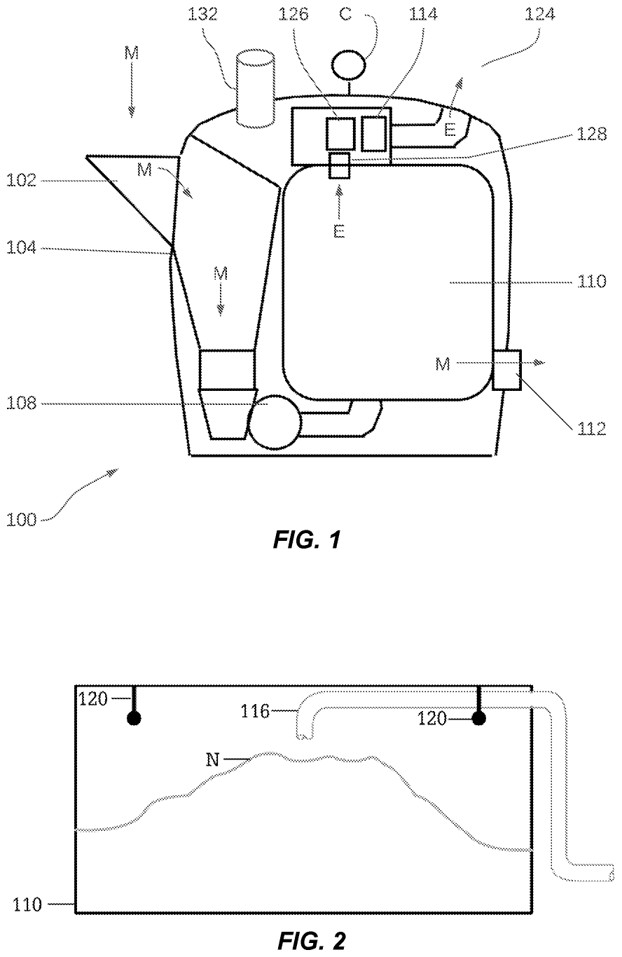

FIG. 1 shows an embodiment of a device 100 for collecting, storing and transporting fermentable waste.

The device 100 is removable and movable between a location for collecting waste and a location for discharging collected waste.

To this end, the device 100 includes, in a flow direction M of the fermentable waste:
a device 102 for delivering fermentable waste;
a hopper 104;
a device 108 for transferring the materials received in the hopper;
a sealed storage tank 110 arranged to store the transferred materials; and
a device 112 for emptying the tank, which device is arranged to empty the storage tank 110.

The device 100 is additionally equipped with a gripping hook C.

The hopper is arranged to direct the fermentable waste from the delivery device 102 to the transfer device 108.

The hopper 104 may be provided with a nonstick coating, for example, of the PTFE or PP (polypropylene) type. In particular, polypropylene has a very low coefficient of friction and good resistance to acids and bases.

The delivery device 102 is of the gravity type.

The delivery device 102 may include a manual opening, preferably with a slide or a drum. Alternatively, or in addition, the delivery device may comprise a container lifter, preferably electrically or hydraulically actuated, arranged to empty a rolling bin.

The device 100 may additionally include two odor traps (not shown).

The first odor trap is formed by a cover of the hopper 104 provided with a gasket and is thus arranged upstream of the hopper, in the flow direction of the materials. The cover has at least two functions, one of which is safety, the other of which is sealing of the hopper. When closed, the hopper cover retains the odors from the residues of waste that has not been conveyed into the storage tank 110.

The second odor trap is formed by a knife gate valve, arranged between the transfer device 108 and the storage tank 110. The knife gate valve ensures that the stored materials cannot return to the hopper 104. This assembly is particularly advantageous when the transfer device has a gooseneck pipe.

The transfer device 108 is arranged to directly transfer the materials received from the hopper to the storage tank 110.

The transfer device is under positive pressure in the flow direction of the waste. The transfer device 108 may be, for example, a compacting worm, also called Archimedes screw, or a peristaltic pump.

In the context of a device 100 including a compactor, for example, when the transfer device 108 also has the function of compacting the materials received in the hopper, the compression applied by the transfer and compacting device may typically be on the order of several tons.

In the embodiment shown, the storage tank 110 is made of polymer, soft metal or stainless steel material. The storage tank may be covered, at least partially, with a protective and/or reinforcing layer, for example, of the PTFE (polytetrafluoroethylene), stainless steel or epoxy sheet type.

According to one possibility, the device 100 may further include an effluent filtering device 114 for the effluent (E), which is fluidly connected to the storage tank 110. Gases can circulate from the storage tank 110 to the outside of the device 100 through the effluent filtering device 114.

According to one embodiment shown in FIG. 2, the transfer device 108 may comprise a gooseneck pipe or cylinder 116 connected above the tank 110.

The storage tank 110 additionally includes level sensors 120.

Curve N shows a possible spread of the materials stored in the storage tank 110.

The emptying device 112 operates by gravity or suction, for example, by way of a rear gate or an agricultural valve.

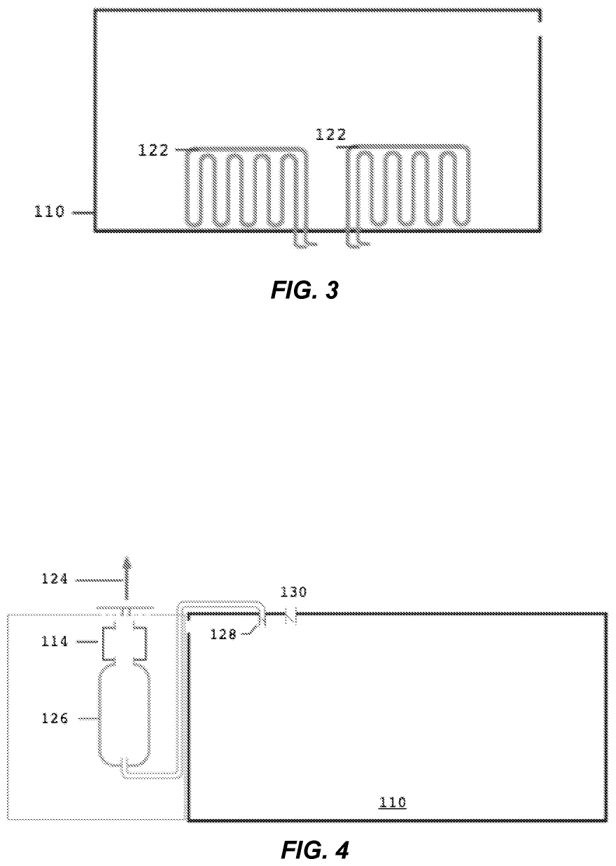

According to one embodiment shown in FIG. 3, the storage tank 110 may further include heating means 122 arranged to heat the stored materials.

Thus, it is possible to heat the storage tank 110 once full to a minimum temperature of 70° C. for a minimum period of one hour in order to carry out the phase of hygienization of the fermentable waste. This phase is required for fermentable waste that may contain type C3 animal by-products (SPAN C3). Performing this operation in the tank subsequently allows the tank to be emptied at any biogas plant, regardless of its facilities. Indeed, since the cost of a hygienization facility in a biogas plant is high, few are equipped with one. Furthermore, a biogas plant that mainly uses agricultural products cannot make a hygienization facility profitable.

It is thus possible to propose a method for collecting, storing and transporting fermentable waste including a step of hygienization of the fermentable waste. The hygienization step being carried out by the heating means 122, for example, at a minimum temperature of 70° C. for a minimum period of one hour.

The hygienization step may be implemented once the filling rate of the storage tank exceeds a predetermined level, more preferentially when the tank is full. For example, it is possible to have level sensors arranged on the tank in a particular position. Other technical solutions are known to a person skilled in the art to determine the filling level of a tank.

Control devices, such as computers, may be provided to control the heating means 122.

According to one embodiment shown in FIG. 4, the effluent filtering device 114 is arranged (fluidly connected) between the storage tank 110 and an effluent discharge device 124, for effluent generated by the stored materials and/or fermentable waste, of the device 100.

The effluent filtering device 114 is a solid filtration medium, preferably a charcoal filter or a biofilter.

The effluent filtering device includes a pressure relief valve 128, arranged between the storage tank 110 and the effluent filtering device 114, preferably calibrated at 0.1 bar.

In the example shown, the storage tank 110 further includes a safety valve 130 calibrated at 0.3 bar at the interface between the storage tank 110 and the outside of the device 100.

The pressure relief valve 128 and the safety valve 130 are arranged as far as possible from the agricultural valve or from the manhole.

For example, the agricultural valve or the manhole can be arranged on one side, called the front side, of the device 100, while the valve 128 and the valve 130 are arranged on the other side, called the rear side, of the device 100.

As shown in FIG. 4, the device 100 may include a pressure reducer 126, arranged between the storage tank 110 and the effluent filtering device 114.

In this case, the pressure relief valve 128 may be arranged between the storage tank 110 and the effluent filtering device 114.

The pressure reducer 126 is chosen carefully and has a volume greater than or equal to the maximum amount of waste ingestible by the machine in a cycle, for example, 240 L. Thus, it is ensured that the effluent filtering device 114 operates at zero pressure. The effluent filtering device 114 thus has the best filtration flow rate.

According to one possibility, the device 100 may further include an effluent storage cylinder 132 fluidly connected to the storage tank 110. The cylinder 132 is, for example, removably mounted on a connector provided on the storage tank 110. Of course, a compressor may be arranged between the storage tank 110 and the cylinder 132.

The cylinder 132 may be equipped with a means for automatically closing its tank when the latter is removed from the storage tank.

The contents of the tank may also be emptied subsequently, according to techniques known to a person skilled in the art.

The cylinder 132 may be connected to the storage tank via the pressure relief valve 128.

When the device 100 may not comprise the effluent filtering device 114.

The decomposing waste gives off CO2; the cylinder allows storage of the CO2 and limitation, if not elimination, of the release of CO2 inherent to the decomposition of the fermentable waste, by not discharging CO2 into the atmosphere.

Of course, the present disclosure is not limited to the examples that have just been described and numerous modifications can be made to these examples without departing from the scope of the present disclosure. In addition, the different features, forms, variants and embodiments of the present disclosure may be associated with one another in various combinations insofar as they are not incompatible or exclusive of one another.

1. A removable device for collecting, storing and transporting fermentable waste comprising, in a flow direction of the waste:
a device for delivering fermentable waste;
a hopper;
a device for transferring materials received in the hopper;
a sealed storage tank arranged to store the transferred materials and operating anaerobically in a storage configuration, and arranged to receive transferred materials in a filling configuration;
a device for emptying the tank, which device is arranged to empty the storage tank; and
an effluent filtering device connected to the storage tank, the effluent filtering device comprising a solid filtration medium;
wherein the hopper is arranged to direct the waste from the delivery device to the transfer device;
the transfer device is arranged to directly transfer the materials received from the hopper to the storage tank; and
the transfer device is under positive pressure in the flow direction of the waste.

2. The removable device of claim 1, further including a cylinder for storing the effluent connected to the storage tank.

3. The removable device of claim 2, wherein the transfer device further comprises a compacting device.

4. The device of claim 3, wherein the transfer device comprises a worm or a peristaltic pump.

5. The device of claim 4, further comprising an odor trap arranged between the transfer device and the storage tank.

6. The device of claim 5, wherein the odor trap comprises a knife gate valve.

7. The device of claim 6, wherein the storage tank further includes a heater arranged to heat the stored materials.

8. The device of claim 7, further comprising a pressure reducer arranged between the storage tank and the effluent filtering device.

9. A method for collecting, storing and transporting fermentable waste implementing a device according to claim 1, the method comprising hygienization of the fermentable waste.

10. The method of claim 9, wherein the hygienization is implemented when the filling rate of the storage tank is higher than a predetermined rate.

11. The removable device of claim 1, wherein the transfer device further comprises a compacting device.

12. The device of claim 1, wherein the transfer device comprises a worm or a peristaltic pump.

13. The device of claim 1, further comprising an odor trap arranged between the transfer device and the storage tank.

14. The device of claim 16, wherein the odor trap comprises a knife gate valve.

15. The device of claim 1, wherein the storage tank further includes a heater arranged to heat the stored materials.

16. The device of claim 1, further comprising a pressure reducer arranged between the storage tank and the effluent filtering device.
